(19) United States
(12) Patent Application Publication
(10) Pub. No.: US 2003/0109909 A1
(43) Pub. Date: Jun. 12, 2003

(54) Tapered vessel radiotherapy

(76) Inventors: Michelle N. Ledesma, Houston, TX (US); Anthony J. Bradshaw, Duluth, GA (US); Eric D. Peterson, Fremont, CA (US); Edward W. Milner JR., Mountain View, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 09/752,182

(22) Filed: Dec. 29, 2000

(51) Int. Cl. A61N 5/00

(52) U.S. Cl. 607/100

(57) ABSTRACT

A method of treating a tapered vessel with radiation. Separate sections of the vessel are treated for independently determined dwell times. A proximal diameter, a distal diameter, and an intermediate diameter can be established upon which prescription points can be based.

### BACKGROUND OF THE INVENTION

The present invention relates to intravascular radiotherapy and devices. In particular, the present invention relates to centering devices and radiotherapy protocols.

### BACKGROUND OF THE PRIOR ART

Several catheters and catheter procedures involve radiotherapy treatment of vessel walls. For example, coronary or peripheral angioplasty, may be followed by vascular radiotherapy as further described here.

Angioplasty generally involves the insertion of an angioplasty catheter into the cardiovascular system under local anesthesia. The angioplasty catheter is delivered by way of a guidewire which has been positioned in the artery ahead of time. The angioplasty catheter, having a distensible balloon portion at its distal end, is advanced until the balloon portion is positioned across a stenotic lesion. The balloon portion is then inflated. The inflation of the balloon compresses the stenotic lesion or atherosclerosis in a direction generally perpendicular to the wall of the artery, dilating the lumen of the artery.

A stent, a supporting cage-like tubular structure, may be placed in conjunction with the dilatation of the artery. This is known as direct stenting. Alternatively, the angioplasty procedure can be followed by placement of a stent at the site of the former stenotic lesion. The stent adds structural support to the injured portion of the vessel.

The angioplasty procedure involves the inherent risk of restenosis and/or the formation of blood clots following the procedure. To help avoid restenosis radiotherapy may be administered at the treatment site following the angioplasty procedure.

Radiotherapy is generally delivered to the site of the former stenosis by way of a radiation catheter. The radiation catheter is inserted after the angioplasty catheter is removed and is generally inserted in the same manner as the angioplasty catheter. The radiation catheter also has a distensible balloon portion at its distal end which is positioned at the site of the former stenotic lesion. The balloon is inflated to secure the catheter within the vessel and to provide a degree of centering within the vessel. The balloon may be inflated with a radiation liquid or an alternate form of radiation, such as a source wire with a radioactive distal tip or radiation pellets, provided by advancing the source wire or pellets through the radiation catheter to a position adjacent the former stenotic lesion to provide radiotherapy.

Radiotherapy is provided throughout a treatment area of a vessel. The treatment area encompasses at least the site of the former stenotic lesion, and generally portions of the vessel proximal and distal the site of the former stenotic lesion. Due to the length of the treatment area, it is generally treated in sections. For example, when treatment is by way of a source wire, a distal-most section of the treatment area will be treated for a specified amount of time (i.e. a dwell time), the source wire will be retracted to the next most distal section where treatment will ensue for a second dwell time, and so forth.

While the angioplasty, or direct stenting, procedure has hopefully eliminated (or compressed) any potentially occluding stenosis, the diameter of the vessel is not generally consistent. For example, in the case of coronary arteries, the vessels will be narrower as the radiation catheter travels further distally. The vessel diameter will not generally be consistent throughout the treatment length of the treatment area. In fact, a vessel may taper as much as about 1.4 mm over a treatment length of 40 mm. Furthermore, the extent of the taper is likely to increase as the treatment length increases. Thus, for example, a taper of greater than 1.4 mm would be possible as the treatment length exceeded 40 mm. At one time standard angioplasty intervention in coronary treatment did not exceed about 22 mm and treatment lengths were of about 27 mm. In such past procedures, utilizing treatment lengths of about 27 mm, vessel tapering was not of major concern. However, this is no longer the case. As conventional practice moves toward larger treatment areas, the likelihood of greater taper sizes increases. Additionally, where treatment is applied to peripheral vasculature (i.e. in non-coronary treatments), both treatment lengths and taper sizes are generally even larger.

In spite of vessel tapers, currently, a uniform vessel profile is presumed when establishing a radiotherapy protocol for use with a uniformly shaped balloon. For example, this hypothetical uniform vessel is given dimensions based on either the diameter of the vessel at the center of the lesion, or by averaging the proximal and distal diameters at the proximal and distal ends of the treatment area. In either case, the presumption of a uniform vessel does not adequately account for the fact that certain sections of the treatment area will be in closer proximity to the radiation source than others due to a vessel taper. Sections such as this may end up with too much radiation, while sections further from the radiation source may not receive enough radiation. If a uniform treatment area is presumed based on the average of these proximal and distal diameters, and about a 1.4 mm taper is present over a 40 mm treatment area, improper dosimetry will occur. The problem of improper dosimetry will be enhanced due to the use of a uniformly shaped balloon that is not reflective of the tapered vessel.

### SUMMARY OF THE INVENTION

An embodiment of the invention provides a method of treating a tapered vessel with radiation. The method involves treating separate sections of the vessel for separate and independently determined dwell times.

In another embodiment a method of radiotherapy is provided accounting for a proximal diameter and a distal diameter of a vessel. An intermediate diameter of the vessel is established. Two prescription points based on the proximal, distal and intermediate diameters.

An alternate embodiment of the invention provides a radiotherapy catheter to treat a tapered vessel. The catheter couples to a radiation source for treating separate sections of the vessel for independently determined dwell times.

An embodiment of a radiotherapy system is provided having a radiation catheter for treatment of a tapered vessel. The system includes an instrument to direct a radiation source to treat separate sections of the vessel for independently determined dwell times.

A radiotherapy system is provided with a tapered balloon catheter for treating a tapered vessel. A radiation source is provided and directable to treat separate sections of the vessel for independently determined dwell times by an instrument.

### BRIEF DESCRIPTION OF THE DRAWINGS

FIG. 1 is a side sectional view of a tapered expansive region of a radiotherapy catheter shown within a tapered vessel.

FIG. 2 is a side view of a tapered expansive region of a radiotherapy catheter within a tapered vessel.

FIG. 3 is a side cross-sectional view of an expansive region of a radiotherapy catheter within a tapered vessel.

FIG. 4 is a chart depicting a dose distribution of an embodiment of the invention throughout a treatment length of a vessel in comparison to a dose distribution of the prior art.

FIG. 5 is a chart depicting a dose distribution of an embodiment of the invention throughout a treatment length of a vessel in comparison to an alternate dose distribution of the prior art.

FIG. 6 is a chart depicting a dose distribution of an alternate embodiment of the invention throughout a treatment length of a vessel in comparison to alternate dose distributions of the prior art.

### DETAILED DESCRIPTION OF THE INVENTION

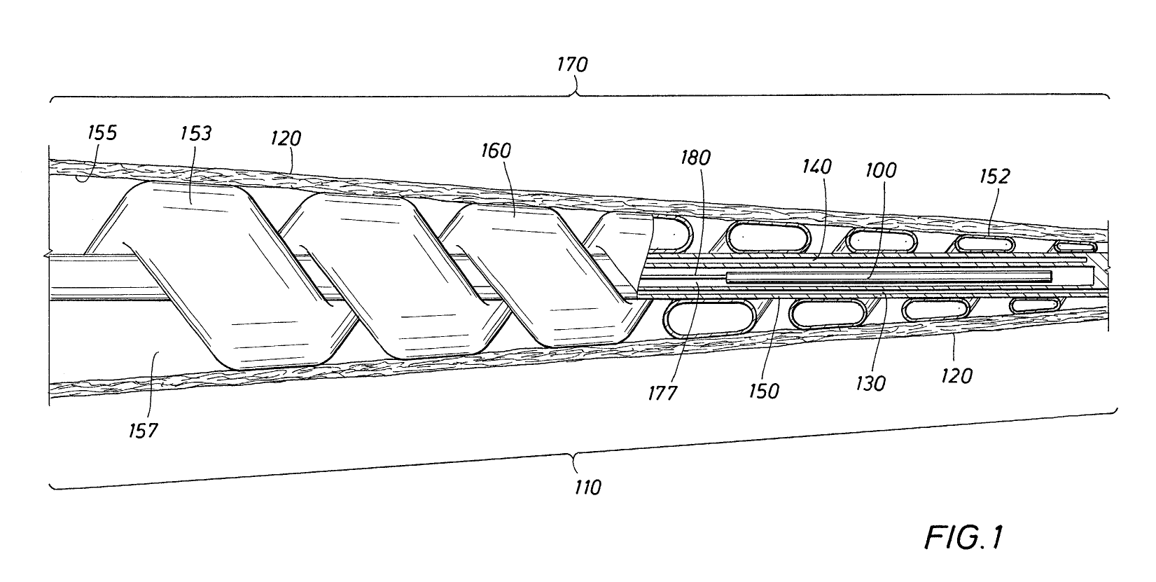

Referring to FIG. 1 an embodiment of a tapered expansive region, in the form of a tapered balloon 160 of a radiotherapy catheter is shown within a tapered vessel 120. For example, in FIG. 1, one end of the vessel 120 has a diameter of 2.1 mm while the other end has a diameter of 3.5 mm to which the tapered balloon 160 is compliant. However, embodiments of the tapered balloon 160 are applicable to various other tapered dimensions. Additionally, the tapered vessel 120 is a coronary tapered vessel 120. However, in an alternate embodiment, the tapered vessel 120 is a peripheral (i.e. non-coronary) tapered vessel 120.

The tapered balloon 160 surrounds a shaft 150 running centrally through the vessel 120. The shaft 150 includes a source wire lumen 177 running there through to accommodate a source wire 180 having a radioactive source 100 at a distal portion thereof. In alternate embodiments alternate forms of radiotherapy are provided through the lumen 177. For example, the lumen 177 can be configured to deliver radiotherapy via radioactive pellets, radiation liquid, and other sources of radiation.

An inflation lumen 140 to inflate the tapered balloon 160 and a guidewire lumen 130 for accommodating a guidewire are also provided through the shaft 150 in an embodiment of the invention. The tapered balloon 160 is secured to the shaft 150 at proximal and distal portions thereof. In an alternate embodiment, the tapered balloon 160 is secured to the shaft 150 throughout the length of the tapered balloon 160 as it contacts the shaft 150.

The tapered balloon 160 of the embodiment shown is configured to deliver radiotherapy while also allowing blood to perfuse past the tapered balloon 160 within the vessel 120. The tapered balloon 160 is spiraled in a manner allowing blood to perfuse between threading of the tapered balloon 160. As a result, the vessel 120 is not occluded during a radiotherapy procedure.

The tapered balloon 160 is also configured to center the shaft 150 within the vessel 120 by being tapered in substantially the same manner as the vessel 120 itself. That is, the tapered balloon 160 has a centering portion 170 which is about the length of the treatment area 110 and tapered throughout. As a result, the shaft 150 and hence, the radioactive source 100, remain centered with respect to the vessel 120 during radiotherapy for delivery of a more evenly distributed radiation dose to the treatment area 110 of the vessel 120. The inflation size of the tapered balloon 160 is selected based on the size and dimensions of the vessel 120 to be treated.

Therefore, because the vessel diameter becomes smaller as the vessel 120 travels distally, so does the tapered balloon 160. This helps prevent a tighter fit that would result at its distal portion 152 by the vessel wall 155. Such a tight fit could risk injury to the vessel 120 in this area. Similarly, as the vessel diameter becomes larger as the vessel 120 travels proximally, so does the tapered balloon 160. This helps prevent a loose and insecure fit that would result at its proximal portion 153 within the vessel lumen 157.

In various embodiments of the invention, various tapered balloon 160 sizes and configurations are used. For example, in one embodiment of the invention, a tapered balloon 160 is provided having the length of the treatment area 110 as mentioned above. In other embodiments, tapered balloon 160 lengths may be from about 20 mm to about 80 mm, and include lengths of 32 mm, 50 mm and 52 mm. With reference to balloon diameter, from one end of the tapered balloon 160 to the other, tapered balloon 160 embodiments include tapers of up to about 2.0 mm, including 0.5 mm, 1.0 mm and other tapers over a given length. In one embodiment a 2.5 mm distal diameter/3.5 mm proximal diameter tapered balloon 160 is provided. In another embodiment a 2.5 mm distal diameter/3.0 mm proximal diameter tapered balloon 160 is provided. Embodiments of the invention employ tapered balloons 160 having distal diameters as small as 2.0 mm while other embodiments employ tapered balloons 160 having proximal diameters as large as 4.0 mm.

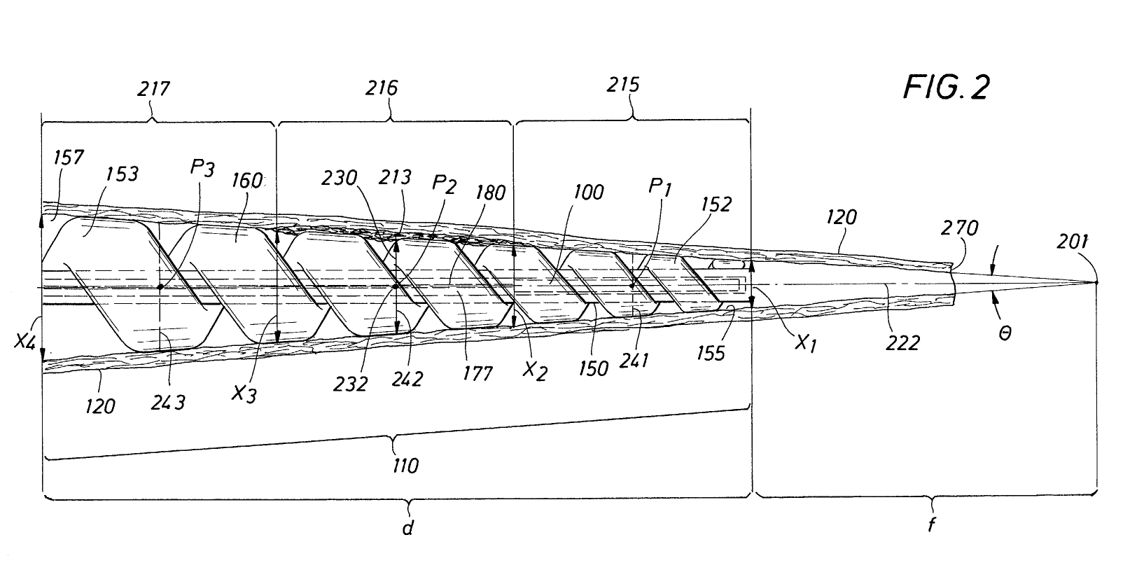

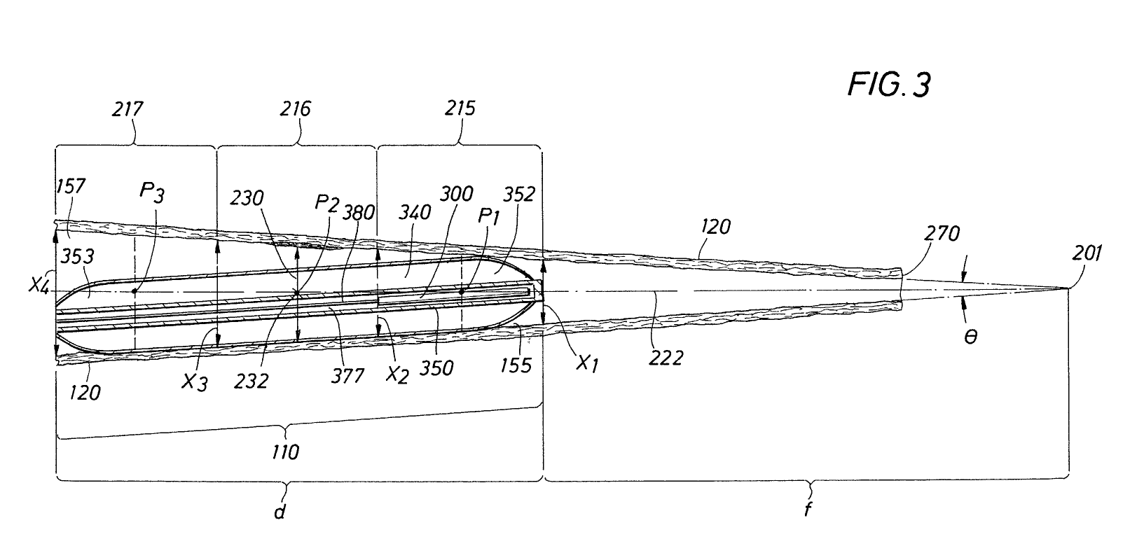

Referring to FIGS. 2 and 3 methods of the invention are shown utilizing a tapered balloon 160 (FIG. 2) and a non-tapered balloon 340 (FIG. 3). In both methods shown, radiotherapy is delivered throughout a treatment area 110 in a more uniform manner.

As shown in FIG. 2, the tapered balloon 160 and source wire 180 have been advanced to the treatment area 110 of the vessel 120. The tapered balloon 160 has a length equivalent to the treatment area 110 in the embodiment shown. However, in alternate embodiments other tapered balloon 160 lengths are utilized. For example, in one alternate embodiment, the tapered balloon 160 covers only a portion of the treatment area 110 and requires multiple adjacent treatments to complete the radiotherapy procedure along the entire treatment area 110. That is, in this alternate embodiment, the tapered balloon 160 will be placed in one portion of the treatment area 110, radiotherapy provided, and the tapered balloon 160 withdrawn or advanced to another portion of the treatment area 110 to provide added radiotherapy.

In the embodiment shown in FIG. 2, the treatment area 110 encompasses portions of the vessel 120 proximal and distal of a former stenosis 213 (or lesion). In one embodiment, the treatment area 110 corresponds to 50 mm of treatment length (d) as measured through the center of the vessel 120. However, alternate embodiments employ treatment areas 110 of anywhere between about 20 mm and about 80 mm, including 32 mm, 52 mm, and other lengths. In the embodiment shown, the treatment area 110 is divided into a first section 215, an intermediate section 216, and a last section 217, each section being one third of the treatment length (d). In the embodiment shown each section 215, 216, 217 is 16.667 mm in length (i.e. one third of the 50 mm treatment length (d) shown). However, in other embodiments alternate treatment and section lengths are utilized. Additionally, in one embodiment of the invention a multiple number of intermediate sections 216 are utilized, while in yet another embodiment, no intermediate sections 216 are utilized.

In a method of the invention treatment is provided throughout the treatment area 110 by radiation emitted from the radioactive source 100. In alternate embodiments radiation is provided via radiation pellets and other sources. In the embodiment shown the radioactive source 100 shown is 16.667 mm in length, corresponding to the length of each section 215, 216, 217 of the treatment area 110. However, in alternate embodiments alternative lengths are utilized. In the method shown treatment is begun by advancing the source wire 180 to the first section 215 at the distal-most portion of the treatment area 110. The source wire 180 and radiation catheter 1 are positioned so that the radioactive source 100 is adjacent the first section 215. Radiation is emitted from the radioactive source 100 for a specified period of time. This amount of time is referred to as dwell time. Once the dwell time is complete, the first step of treatment is complete. Next, the radioactive source 100 is withdrawn proximally until it is adjacent the intermediate section 216 and the second step of treatment is begun in the same manner as the first (i.e. for a second dwell time). Lastly, a third step of radiation treatment will be provided to the last section 217.

Each section 215, 216, 217 of the treatment area 110 is to absorb a specific amount of radiation. For example, in one embodiment, between about 16 to 24 Grays (1600-2400 rads), as measured from 1 mm. of depth into tissue of the vessel 120, is preferably absorbed. Achieving this absorption will depend upon dwell time and how far the vessel 120 is from the radioactive source 100 among other factors.

Embodiments of the present invention utilize independently determinable dwell times for each step of treatment at each section 215, 216, 217. The dwell time for each step is correlated to a prescription point (P₁, P₂, P₃) which is particular to each section 215, 216, 217. For example, in the embodiment shown, the first section 215 is defined by the distal diameter (X₁) and a first intermediate diameter (X₂). A first prescription diameter 241 is located half way between the distal diameter (X₁) and the first intermediate diameter (X₂). One half of this first prescription diameter 241 gives the location of the first prescription point (P₁) (i.e. its distance from the vessel 120). The dwell time for the first step is based on the first prescription point (P₁) which is found in the first section 215 irrespective of the proximal diameter (X₄) or the location of the former stenosis 213.

The intermediate section 216 of the embodiment shown is defined by the first intermediate diameter (X₂) and a second intermediate diameter (X₃) with an intermediate prescription diameter 242 there between. The intermediate prescription point (P₂) is one half of the intermediate prescription diameter 242 from the vessel 120. Again, the dwell time for the second step is based on the intermediate prescription point (P₂) which is found within the intermediate section 216. This dwell time is determined irrespective of the proximal diameter (X₄), the distal diameter (X₁), or the location of the former stenosis 213. In alternate embodiments, where different numbers of intermediate sections are utilized, dwell times are still determined independently.

In the embodiment shown, a treatment area 110 utilizing an odd number of sections 215, 216, 217 is utilized. In embodiments of the invention where this is the case, an intermediate prescription diameter 242 is approximately aligned with the center of the former stenosis 213.

Referring again to the embodiment of FIG. 2, the last section 217 is defined by a last intermediate diameter (X₃) and the proximal diameter (X₄) with a last prescription diameter 243 there between. In this embodiment last intermediate diameter (X₃) is also the second intermediate diameter (X₃) because there is only one intermediate section 216. The third prescription point (P₃) is located half of the prescription diameter 243 from the vessel 120. The dwell time for this last step is based on the third prescription point (P₃) which is found within the last section 217. Again, the dwell time for the last section 217 is determined irrespective of the distal diameter (X₁) or the location of the former stenosis 213.

As indicated above, the location of the prescription point (P₁, P₂, P₃) helps to determine a separate dwell time for each step of treatment. Given a predetermined number of treatment steps and a known treatment length (d), each of the prescription points (P₁, P₂, P₃) is determined by measuring the proximal diameter (X₄) and distal diameter (X₁).

It is noted that, beginning with the distal diameter (X₁), each diameter of interest is numbered (i.e. Xₙ) from right to left as shown. This holds true in embodiments of the invention, regardless of the number of sections utilized during a given treatment. Therefore, in connection with the following equations, the distal diameter (X₁) will always be noted as “X₁” but the proximal diameter (X₄) will not be noted as “X₄” unless the treatment area 110 is divided into three sections 215, 216, 217, as in the embodiment of FIG. 2.

In order to determine the location of the prescription points (P₁, P₂, P₃), the vessel 120 is approximated as a cone 270 terminating in a hypothetical fulcrum 201. The angle φ of this cone 270 is indicated according to equation 1, where n equals the total number of sections to be treated. Values for the diameters (X) are determined by conventional means. For example, embodiments of the invention make use of techniques such as angiography or ultrasound to determine diameter sizes.

$$φ = {tan}^{- 1} ( \frac{{X}_{n + 1} - {X}_{1}}{d} ) \qquad Equation 1$$

The angle φ of the cone 270 is used to determine a fulcrum distance (f). The fulcrum distance (f) is the distance between the distal diameter (X₁) and the fulcrum 201 as measured through the vessel center 222. The fulcrum distance (f) is given by equation 2:

$$f = ( \frac{{X}_{1}}{tan φ} ) \qquad Equation 2$$

Given values for the angle φ of the cone 270 and the fulcrum distance (f), the size of each intermediate diameter (X₂, X₃) is determined according to equation 3, where i equals the diameter sought (right to left in FIG. 2):

$${X}_{i} = ( \frac{i - 1 ) d}{n} + f ) tan φ \qquad Equation 3$$

Once values have been established for the intermediate diameters (X₂, X₃), the prescription point (P₁, P₂, P₃) locations are determined according to equation 4, where i equals the prescription point sought (right to left in FIG. 2):

$${P}_{i} = \frac{1}{2} ( \frac{{X}_{i} + {X}_{i + 1}}{2} ) \qquad Equation 4$$

Equation 4 has now given the distance between the vessel 120 and each separate prescription point (P₁, P₂, P₃) upon which independent dwell times for each step of treatment may be based. In an embodiment of the invention, the dwell time will be based on this distance plus 1 mm. This is because, treatment and measurements are to be based upon 1 mm of vessel 120 depth in this embodiment. Thus, in the described embodiment for example, the first section 215 will have a dwell time based directly on the value of the first prescription point (P₁)+1. For clarity reference is now made to adjusted prescription points (A₁, A₂, A₃) where A₁=P₁+1, A₂=P₂+1, and A₃=P₃+1.

For illustrative purposes an embodiment of the invention utilizing a 16.667 mm, 100 millicurie radiation source 200 is now considered. The source 200 is to provide 20 Grays of radiation at 1 mm. of tissue depth throughout the treatment area 110. Now that there is an adjusted prescription point (A₁, A₂, A₃) a dwell time (T₁, T₂, T₃, numbered starting with the first section 215) is determinable.

Based on actual studies, embodiments of the present invention make use of a dose rate table, where delivery of each Gray of radiation is predetermined. The dose rate may be given in Grays/seconds/millicurie (Gy/s/mCi) for any given adjusted prescription point (Aᵢ). For example, given a 100 mCi radiation source 200 and a dose rate of 0.002 Gy/s/mCi, we know that it will take 100 seconds of dwell time (Tᵢ) to deliver a prescribed dose of 20 Grays to the section 215, 216, or 217 having our given adjusted prescription point (Aᵢ). That is:

$$\frac{20 {G}_{y}}{( 0.002 G y \text{/} s \text{/} m C i ) ( 100 m C i )} = 100 s \qquad Equation 5$$

Now that dwell times for each section 215, 216, 217 are determined, a dose plan accounting for all three dwell times (T₁, T₂, T₃) is provided. This entire dose plan has been developed from the mere measurement and input of the proximal diameter (X₄) and the distal diameter (X₁). Once the measurements and determinations of equation 1 (i.e. X₁, Xₙ₊₁, n, and d) and the dose rate table have been established, the remainder of the process is performed by way of an automated afterloader system.

Referring to FIG. 3, a method of the invention is applied making use of a non-tapered balloon 340. The non-tapered balloon 340 is not securely positioned at its proximal portion 353. Rather, during a radiotherapy procedure, it is prone to movement within the tapered vessel 120 away from the vessel center 222. For example, during a portion of a radiotherapy procedure, the non-tapered balloon 340 can even be positioned as shown, against the vessel wall 155. Nevertheless, the method described with reference to FIG. 2 can still be applied utilizing a non-tapered balloon 340. In fact, the method of the invention described above with reference to FIG. 2 can be applied using a non-tapered balloon 340 and continue to deliver a more uniform radiation dose throughout the treatment area 110 as described further herein.

In the embodiment of FIG. 3, a shaft 350 runs through the non-tapered balloon 340 in order to provide stability to the non-tapered balloon 340 and a source wire lumen 377. A source wire 380 having a radioactive source 300 is shown advanced to the non-tapered balloon 340 via the source wire lumen 377.

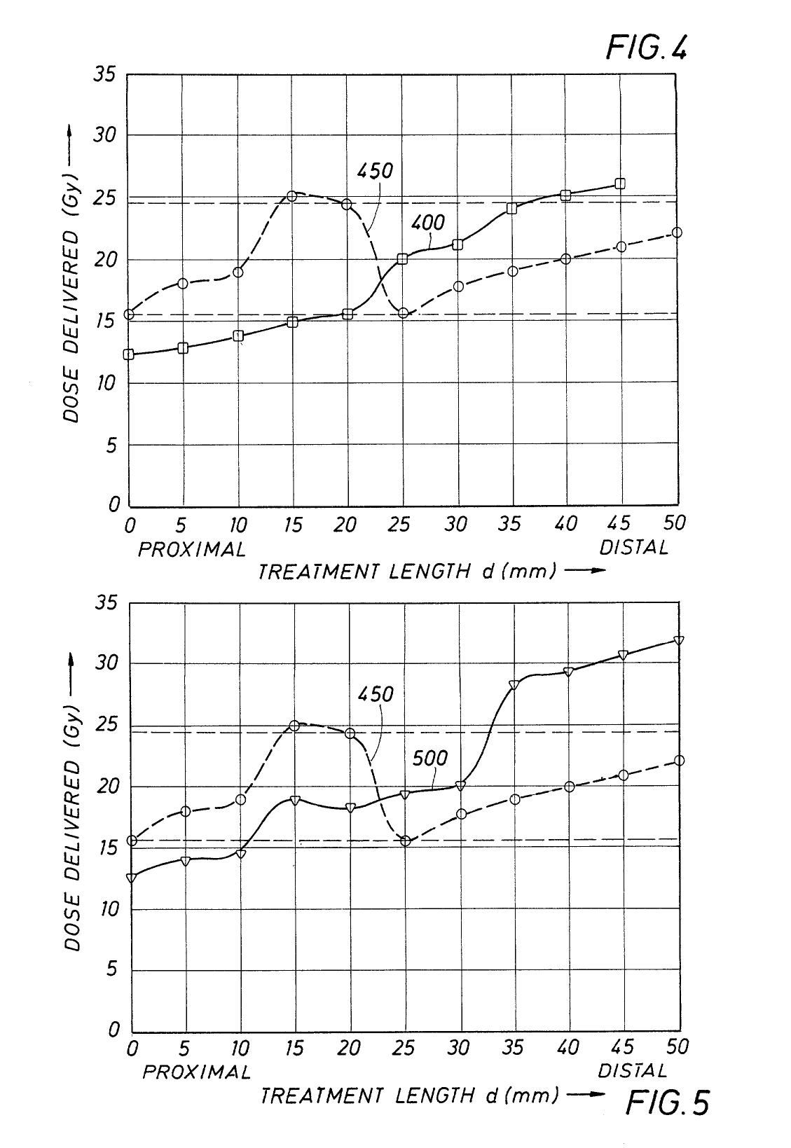

With reference to FIGS. 4 and 5 a dose distribution 450 of a method of radiotherapy according to an embodiment described above is shown in comparison to a prior art dose distribution 400. The dose distribution 450 shown is achieved making use of a non-tapered balloon 340 (see FIG. 3). However, other dose distributions are obtained in alternate embodiments of the invention making use of a tapered balloon 160 (see FIG. 2) as discussed further herein.

Referring to FIG. 4, the prior art dose distribution 400 utilizes a single dwell time based on a stenotic point 232, which in this case happens to correspond with the second prescription point (P₂) (see FIGS. 2 and 3). However, where alternate methods of the present invention are employed, this will not be the case. In the prior art dose distribution 400 method of radiotherapy each step of treatment at each section 215, 216, 217 will utilize the same dwell time while the radioactive source 100, 200, 300 emits a consistent amount of radiation.

As mentioned earlier, the amount of radiation absorbed by the vessel 120 in each section 215, 216, 217 depends upon the dwell time and how far the vessel 120 is from the radioactive source 100, 200, 300 (see FIGS. 1-3). Because the vessel 120 is tapered and not of a consistent diameter the distance between the radioactive source 100, 200, 300 and the vessel 120 is not consistent. As a result, each section 215, 216, 217 does not absorb radiation at the same rate nor in the same amount if only one dwell time is used for all sections 215, 216, 217. Therefore, where a single dwell time is used as in the prior art dose distribution 400 method referenced in FIG. 4, distribution of dose delivery throughout a given treatment length (d) will vary significantly.

The prior art dose distribution 400 method referenced in FIG. 4 presumes that the treatment area 110 has a consistent diameter which is equivalent to a stenotic diameter 230, which, as shown in FIGS. 2 and 3, corresponds to the intermediate prescription diameter 242. It is further presumed that the midpoint of the stenotic diameter 230, the stenotic point 232, is where the radioactive source 100, 200, 300 will be located. Thus, determining the position of the stenotic point 232, its distance from the vessel 120, is determinative of the single dwell time for all sections 215, 216, 217.

With continued reference to the chart of FIG. 4, the amount of radiation delivered (in Grays) over a 50 mm treatment length (d) having a 1.4 mm taper is shown. The prescribed dose of radiation throughout the treatment area is between 16 and 24 Grays. As the chart shows, an overdose of radiation occurs near the distal end of the treatment area while an under-dose is delivered to the proximal end where a prior art dose distribution 400 is referenced. In fact, only 15 mm of treatment area (between 20 and 35 mm from the proximal end) receive the prescribed dose of radiation pursuant to the prior art dose distribution. This 15 mm of proper treatment will not increase as radiotherapy proceeds. That is, if treatment were provided further distally, the same or additional overdosing would occur and if treatment were provided further proximally, the same or additional under-dosing would occur.

FIG. 4, as mentioned above, also shows a dose distribution 450 of a method of radiotherapy according to an embodiment of the invention utilizing separate dwell times for each step of treatment based upon independent prescription points (P₁, P₂, P₃). With the exception of a short segment of treatment area near the 15 mm point, the entire treatment area, ranging nearly 50 mm in treatment length (d), receives the prescribed dose of radiation where a method of radiotherapy of the present invention is employed (as seen at dose distribution 450). In fact, the dose does not exceed 25 Grays at any point. This is in sharp contrast to the prior art dose distribution 400 which reflects a delivery of the prescribed dose to only about 15-20 mm. of treatment length (d).

With reference to FIG. 5 the dose distribution 450 described with reference to FIG. 4 is again shown. Again, the chart shows the amount of radiation delivered (in Grays) over a 50 mm treatment length (d) having a 1.4 mm taper. The prescribed dose of radiation throughout the treatment area is between 16 and 24 Grays.

In FIG. 5, the dose distribution 450 is shown in comparison to a second prior art dose distribution 500. The second prior art dose distribution 500 again utilizes a single dwell time based on a stenotic point 232 (see FIGS. 2 and 3). However, instead of directly determining a stenotic point 232 in line with the site of the former stenosis 213, the proximal diameter (X₄) and the distal diameter (X₁) are averaged to give a diameter average. The stenotic point 232 is presumed based on this diameter average. Dwell time for each step is again based on the location of this single stenotic point 232, as determined from the diameter average.

In spite of making use of the above referenced diameter average and stenotic point 232 the chart of FIG. 5 reveals that the second prior art dose distribution varies significantly outside of the prescribed dose range of between 16 and 24 Grays. Again an overdose of radiation occurs near the distal end of the treatment area while an under-dose is delivered to the proximal end. This time slightly more than 15 mm of treatment area (between 15 and 30 mm from the proximal end) receive the prescribed dose of radiation. However, a greater degree of overdose occurs near the distal end of the treatment area. Furthermore, as the treatment area increases the degree of under and overdosing become greater.

FIG. 5, as mentioned above, again shows a dose distribution 450 of a method of radiotherapy according to an embodiment of the invention utilizing separate dwell times. Utilizing separate dwell times again provides a more consistent dose distribution 450 throughout a treatment length (d). Regardless of the prior art method chosen, if a single dwell time is utilized, a less consistent dose distribution 400, 500 will result (see also FIG. 4). Furthermore, as treatment areas begin to exceed 50 mm in length the degree of improper dosing will continue to increase with use of prior art methods resulting in prior art dose distributions 400, 500.

In another embodiment of the invention a tapered balloon 160 (see FIG. 1) is used to deliver radiotherapy according to a method of the invention which employs independently determined dwell times. As mentioned above, use of a non-tapered balloon 340 involves a degree of insecurity where the vessel 120 is too large to appropriately center the non-tapered balloon 340. Radiotherapy provided to the vessel 120 is affected by this insecurity as the non-tapered balloon 340 is prone to rest off center from the center of the vessel 120 (see FIGS. 2 and 3). As a result, a larger amount of radiation will be absorbed by portions of the vessel 120 in closer contact with the non-tapered balloon 340. These portions of the vessel 120 will be in closer proximity to the radioactive source 100, 200, 300 as radiotherapy proceeds while other portions will be further distanced receiving less radiation. This affects the dose distribution. However, in an embodiment of the invention, a tapered balloon 160 (as shown in FIG. 1) is used during a radiotherapy procedure that includes independently determinable dwell times.

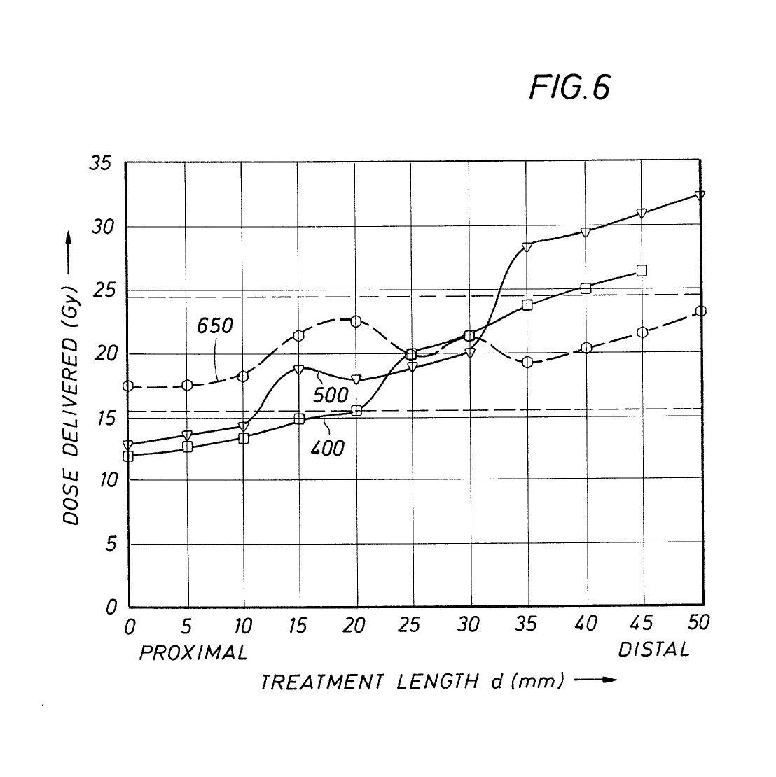

Referring to FIG. 6, a chart is shown depicting a tapered balloon dose distribution 650 resulting from use of a tapered balloon 160 to deliver radiotherapy according to a method employing independently determinable dwell times as described above. Embodiments of the invention described with reference to FIGS. 4 and 5 provide a level of precision to intravascular radiotherapy not previously available to tapered vessels. When combined with a tapered expansive region, such as in the form of a tapered balloon 160 embodiment of the invention, this level of precision increases. In fact, in such an embodiment, where a range of between 16 and 24 Grays of radiation is precribed throughout a tapered vessel, no overdose or underdose is delivered throughout at least about 50 mm of treatment length (d).

Again, prior conventional treatments, reflected by prior art dose distributions 400, 500 deliver an improper dose of radiation to the majority of the treatment area 110 (see FIGS. 1-3). As treatment lengths (d) begin to exceed 50 mm in length the degree of improper dosing will continue to increase with prior art treatment methods. Alternatively, embodiments of the present invention allow for treatment of treatment areas beyond 50 mm with a heretofore unseen accuracy in delivery of a prescribed dose of radiation.

Embodiments of the invention include a tapered balloon for a radiotherapy catheter and methods of use. More accurate radiotherapy treatments are allowed as more accurate measurements and methods are provided. Furthermore, in an embodiment of the invention, true vessel form is more accurately reflected to further increase accuracy in radiotherapy. Although exemplary embodiments of the invention have been shown and described in the form of radiation therapy utilizing source wire radiation and three sections 215, 216, 217, and/or a tapered balloon, many changes, modifications, and substitutions may be made without departing from the spirit and scope of this invention. For example, the present invention would be applicable to any radiation procedure taking place within a tapered lumen. Additionally, radiation may be provided in the form of a fluid, pellets, or any other generally acceptable form.

We claim:

1. A method of treating a tapered vessel with radiation, said method comprising:
providing radiation treatment to a first section of said vessel for a first dwell time; and
supplying radiation to a last section of said vessel for a last dwell time, said first dwell time and said last dwell time determined independently.

2. The method of claim 1 further comprising radiating a plurality of intermediate sections of said vessel for intermediate dwell times, each said intermediate dwell time determined independently.

3. The method of claim 1 wherein said treating is applied to a treatment area of said vessel, said treatment area including a site of a former stenosis.

4. The method of claim 1 further comprising radiating an intermediate section of said vessel for an intermediate dwell time, said intermediate dwell time determined independent of said first dwell time and said last dwell time.

5. The method of claim 4 wherein said first section is defined by a distal diameter and a first intermediate diameter, said last section defined by a proximal diameter and a last intermediate diameter.

6. The method of claim 5 wherein said first intermediate diameter and said last intermediate diameter comprise the same diameter.

7. The method of claim 5 wherein said intermediate section is defined by said first intermediate diameter and a second intermediate diameter.

8. The method of claim 7 wherein said second intermediate diameter and said last intermediate diameter comprise the same diameter.

9. The method of claim 1 wherein said treating is applied to a treatment area of said vessel, said treatment area having a treatment length of up to about 80 mm as measured through a center of said vessel.

10. The method of claim 9 wherein said vessel has a taper of no less than about 0.5 mm throughout said treatment area.

11. The method of claim 9 wherein said providing and said supplying result in said treatment area absorbing at least about 16 Grays at 1 mm. of depth into tissue of said treatment area.

12. The method of claim 9 wherein said providing and said supplying result in said treatment area absorb ing no more than about 25 Grays at 1 mm. of depth into tissue of said treatment area.

13. The method of claim 9 wherein said providing and said supplying result in said treatment area absorbing at least about 16 Grays and no more than about 24 Grays at 1 mm of depth into tissue of said treatment area throughout at least about 45 mm of said treatment length.

14. A method of treating a tapered vessel with radiation, said method comprising:
determining a proximal diameter and a distal diameter of said vessel;
ascertaining at least one intermediate diameter of said vessel;
introducing at least two prescription points based on said proximal diameter, said distal diameter, and said intermediate diameter.

15. The method of claim 14 wherein said ascertaining further comprises establishing an angle of a cone from said proximal diameter and said distal diameter.

16. The method of claim 15 wherein said ascertaining further comprises finding a fulcrum distance, said angle of said cone terminating in a fulcrum, said intermediate diameter being said fulcrum distance from said fulcrum.

17. The method of claim 15 wherein said establishing further comprises selecting a treatment length, said proximal diameter and said distal diameter being separated by said treatment length there between.

18. The method of claim 17 further comprising radiating sections of said vessel with a radiation source.

19. The method of claim 18 wherein said sections are radiated for specified dwell times.

20. The method of claim 19 wherein each said dwell time is based on one of said prescription points.

21. The method of claim 20 further comprising creating a dose rate table.

22. The method of claim 20 wherein said prescription point is adjusted in order to determine said dwell time.

23. The method of claim 20 wherein each said prescription point is related to adjacent diameters of said proximal diameter, said distal diameter, and said intermediate diameter, according to the following:
$${P}_{i} = 1 \text{/2} ( \frac{{X}_{i} + {X}_{i} + 1}{2} )$$
where:
Xᵢ=a first diameter of said adjacent diameters;
Xᵢ₊₁=a second diameter of said adjacent diameters; and
Pᵢ=a prescription point between said adjacent diameters.

24. The method of claim 20 wherein a diameter of said proximal diameter, said distal diameter and said intermediate diameter is related to a section according to the following:
$${X}_{i} = ( \frac{( i - 1 ) d}{n} + f ) tan ∅$$
where:
Ø=said angle;
f=said fulcrum distance;
d=said treatment length;
i=a number associated with said diameter based on its position relative to said fulcrum; and
X₁=said diameter.

25. The method of claim 20 wherein said fulcrum distance is related to said distal diameter according to the following:
$$f = ( \frac{{X}_{1}}{tan ∅ } )$$
where:
f=said fulcrum distance; and
X₁=said distal diameter.

26. The method of claim 20 wherein said angle is related to said proximal diameter and said distal diameter according to the following:
$$∅ = {tan}^{- 1} ( \frac{{X}_{n + 1} - {X}_{1}}{d} )$$
where:
Ø=said angle;
Xₙ₊₁=said proximal diameter;
X₁=said distal diameter; and
d=said treatment length.

27. A radiation catheter to treat a tapered vessel, said catheter to couple to a radiation source to treat a plurality of sections of said vessel for independently determined dwell times.

28. The radiation catheter of claim 27 wherein said radiation source is a radioactive distal tip of a source wire, said source wire insertable through a lumen of said catheter.

29. The radiation source of claim 27 having a length of about a length of each said section as measured through a center of said vessel.

30. A radiotherapy system comprising:
a radiation catheter to treat a tapered vessel with a radiation source;
an instrument to direct said radiation source to treat a plurality of sections of said tapered vessel via said catheter, said instrument including a device to further direct said radiation source to treat each said section for an independently determined dwell time.

31. A radiotherapy system comprising:
a tapered balloon catheter deliverable to a tapered vessel;
a radiation source deliverable to a lumen of said tapered balloon catheter; and
an instrument to direct said radiation source to treat a plurality of sections of said tapered vessel for independently determined dwell times.
